(19) United States
(12) Patent Application Publication
(10) Pub. No.: US 2009/0023714 A1
(43) Pub. Date: Jan. 22, 2009

(54) COMBINED PREPARATION OF A THIAZIDE DIURETIC AND A LOOP DIURETIC

(75) Inventors: Heinrich Knauf, Freiburg (DE); Ernst Mutschler, Mainz (DE)

Correspondence Address: CATALYST LAW GROUP, APC, 9710 SCRANTON ROAD, SUITE S-170, SAN DIEGO, CA, 92121, US

(73) Assignee: HEXAL AG

(21) Appl. No.: 12/065,261

(22) PCT Filed: Aug. 29, 2006
(86) PCT No.: PCT/EP2006/008455

§ 371 Date: Jun. 26, 2008

(30) Foreign Application Priority Data
Aug. 29, 2005 (DE) 10 2005 040 841.9

(51) Int. Cl. A61K 31/5415; A61K 31/4035; A61K 31/517; A61K 31/44; A61P 9/12

(52) U.S. Cl. 514/223.5; 514/417; 514/266.3; 514/347

(57) ABSTRACT

The invention relates to pharmaceutical preparations which contain a combination of a thiazide diuretic and a low-dose loop diuretic and which are particularly useful for treating hypertension and heart failure. It is not only the overall natriuretic/diuretic effects which are increased due to said combination of thiazide diuretic and loop diuretic, but the undesired secondary effects regarding potassium and magnesium losses are significantly reduced as well. As a result, the desired hypocalciuric effect of the thiazide is maintained despite the presence of the loop diuretic.

The present invention relates to pharmaceutical preparations containing a fixed combination of a low-dose loop diuretic and a thiazide diuretic.

Free combinations of loop diuretic/thiazide diuretic were—and are—successfully used in patients with so called diuretic resistance, i.e. in patients where even high doses of a loop diuretic are not or not sufficiently effective, and in patients with severe renal failure. In 1971 it was reported for the first time that, following additional administration of a thiazide diuretic (for example chlorthalidone), diuresis was re-established in patients suffering from severe cardiac disease who did no longer respond to high-dose infusion therapy with loop diuretics (e.g. furosemide). In patients with severe renal diseases, the diuretics combination allows reducing the very high dose of loop diuretics usually required and thus reducing the incidence of secondary effects. However, in both cases it has been noted early in literature (1, 22) that such a treatment, due to the risk of serious disturbances in water and electrolyte balance, should take place only under intensive stationary control of the patients in specially trained medical facilities such as, for example, intensive care units or nephrological centres.

The described combined treatment is based on the principle of sequential nephron blockade. This is defined as the action of said diuretics at different, consecutive segments of the tubular system. Loop diuretics, as their name implies, act on the thick ascending limb of the loop of Henle where they inhibit the Na⁺/K⁺/2Cl⁻ co-transporter, whereas thiazides block the Na⁺/Cl⁺ co-transporter situated more distally at the early distal tubulus. This effect is overproportionally increased by the interaction of the diuretics with the kidney's transporter systems at two different sites (“supra-additive effect”; 2-27).

While in the above described acute treatment of dangerous conditions therapy starts with a high-dose loop diuretic and is continued by additional administration of a thiazide only in case of insufficient response, in permanent treatment of cardiovascular diseases emphasis is given to the administration of a thiazide, as thiazides proved to be more effective than loop diuretics especially in the case of hypertension (28, 29, 30). While it was demonstrated in numerous studies that such a thiazide treatment significantly reduces mortality of patients with hypertension, this treatment nevertheless suffers from disadvantages such as potassium and magnesium losses as well as deterioration of the fat, sugar and uric acid metabolism at higher doses.

These undesired electrolyte losses were first counteracted by oral electrolyte substitutions which, however, were not well tolerated especially by elderly patients, and later by adopting fixed combinations of a thiazide and an antikaliuretic, e.g. amiloride or triamterene. However, this fixed combination also turned out to be problematical because of the risk of hyperkalemia in patients with increased plasma creatinine (as well as in elderly with pseudo-normal plasma creatinine). In addition, co-administration with a thiazide excludes the often required additional treatment with an ACE inhibitor and an AT1 receptor antagonist, respectively.

It has now surprisingly been found that not only is the overall natriuretic/diuretic effect increased by a fixed combination of a thiazide diuretic and a low-dose loop diuretic so that a supra-additive effect is achieved even using this low-dose combination, but that undesired secondary effects regarding potassium and magnesium losses are significantly reduced as well. At the same time, the desired hypocalciuric effect of the thiazide is maintained even in the presence of the loop diuretic.

Thus, the object of the present invention is a combination preparation comprising as active ingredients, jointly or separately from each other:
(a) a thiazide diuretic in an amount corresponding to the effect of a dose of from 5 to 50 mg of hydrochlorothiazide; and
(b) a loop diuretic in an amount corresponding to the effect of a dose of from 2.5 to 15 mg of torasemide.

The combination preparation of the present invention, thus, is a fixed combination of active ingredients, selection and doses of these ingredients being pre-determined.

The combination preparation may contain the active ingredients, which usually are present in solid form, jointly, i.e., in the form of a sole dosage unit, or separately, i.e., in the form of individual dosage units for co-administration.

Thiazide diuretics, which may be used in the combination preparation of the present invention, are understood to be diuretics which inhibit the Na⁺/Cl⁻ carrier in the distal tubulus and increase the excretion of sodium, chloride, potassium and magnesium ions. This includes thiazide and thiazide analogs. Useful thiazide diuretics, for example, are hydrochlorothiazide, chlorothiazide, benzthiazide, cyclothiazide, trichlormethiazide, cyclopenthiazide, methyclothiazide, benzylhydrochlorothiazide, ethiazide, hydroflumethiazide, polythiazide, bendroflumethiazide, chlorthalidone, indapamide, mefruside, metolazone, bemetizide, clopamide, fenquizone, quinethazone, butizide, xipamid and indapamide, and pharmaceutically acceptable derivatives thereof, for example salts thereof. Preferably hydrochlorothiazide is used as a thiazide diuretic.

Loop diuretics, which may be used in the combination preparation of the present invention, are understood to be diuretics which, as mentioned above, inhibit the Na⁺/K⁺/2Cl⁻ carrier in the thick ascending limb of the loop of Henle and thereby inhibit the reabsorption of sodium, potassium and chloride ions. Useful loop diuretics are torasemide, furosemide, azosemide, bumetanide, piretanide, tripamide, etozoline and its metabolite ozolinone, and cicletanine, and pharmaceutically acceptable derivatives thereof, for example salts and esters thereof. Preferably, torasemide is used as a loop diuretic.

The combination preparation of the present invention contains the thiazide diuretic in the respective dosage unit in an amount corresponding to a dose of from 5 to 50 mg of hydrochlorothiazide. More preferably, the thiazide diuretic is present in an amount corresponding to a dose of from 5 to 40 mg of hydrochlorothiazide, and an amount corresponding to a dose of from 10 to 30 mg is most preferred. Typical amounts of the thiazide diuretic correspond, for example, to a dose of 12.5 and of 25 mg of hydrochlorothiazide.

An “amount of a thiazide diuretic corresponding to the effect of a dose of from 5 to 50 mg of hydrochlorothiazide” is understood here to be the amount of a thiazide diuretic which, for the duration of action of this thiazide diuretic, results in the excretion of the same quantity of NaCl as 5 to 50 mg of hydrochlorothiazide. In this context, the term “duration of action” of the thiazide diuretic refers to the period of time within which NaCl excretion induced by the active ingredient is above control excretion without thiazide diuretic (“baseline”). The amount of a thiazide diuretic corresponding to the effect of a dose of 25 mg of hydrochlorothiazide, for example, is an amount which, for the duration of action of this thiazide diuretic, results in excretion of the same quantity of NaCl as 25 mg of hydrochlorothiazide. The efficacy profile of diuretics is described in detail, for example, by Knauf and Mutschler (31).

Accordingly, thiazide diuretics such as hydroflumethiazide, benzthiazide, chlorthalidone and quinethazone are usually used, like hydrochlorothiazide, in an amount of from about 5 to 50 mg, more preferably of from about 5 to 40 mg, and most preferably of from about 10 to 30 mg, for example of from 12.5 to 25 mg per dosage unit. Bendroflumethiazide, metolazone and methyclothiazide are usually present in an amount of from about 0.5 to 5 mg, more preferably of from about 0.5 to 4 mg, and most preferably of from about 1 to 3.0 mg. Indapamide is usually present in an amount of from about 0.25 to 1.25 mg, more preferably of from about 0.25 to 2 mg, and most preferably of from about 0.5 to 1.5 mg. Polythiazide and trichlormethiazide are usually present in an amount of from about 0.2 to 2 mg, more preferably of from about 0.2 to 1.6 mg, and most preferably of rom about 0.4 to 1.2 mg.

The loop diuretic of the combination preparation of the present invention in the respective dosage unit is present in an amount corresponding to the effect of a dose of from 2.5 to 15 mg of torasemide. Preferably, the loop diuretic is present in an amount corresponding to an amount of 2.5 to 14 mg, more preferably an amount of from 2.5 to 12.5 mg. Typical amounts of the loop diuretic correspond, for example, to a dose of 2.5, 5 or 10 mg of torasemide.

An “amount of a loop diuretic corresponding to the effect of a dose of from 2.5 to 15 mg of torasemide” is understood here to be the amount of a loop diuretic which, for the duration of action, of this loop diuretic results in the excretion of the same quantity of NaCl as 2.5 to 15 mg of torasemide. As above, in this context the term “duration of action” refers to the period of time within which NaCl excretion induced by the active ingredient is above control excretion without loop diuretic (“baseline”). For example, the amount of a loop diuretic corresponding to the effect of a dose of 10 mg of torasemide is an amount which, for the duration of action of this loop diuretic, results in excretion of the same quantity of NaCl as 10 mg of torasemide (see also 31).

Accordingly, loop diuretics such as piretanide are usually used, like torasemide, in an amount of from about 2.5 to 15 mg, more preferably of from about 2.4 to 14 mg, and most preferably of from about 2.5 to 12.5 mg per dosage unit. Furosemide is usually present in an amount of from about 7.5 to 45 mg, more preferably of from about 7.5 to 42 mg, and most preferably of from about 7.5 to 37.5 mg, and bumetanide is usually present in an amount of from 0.05 to 0.3 mg, more preferably of from about 0.05 to 0.25 mg, and most preferably of from about 0.05 to 0.2 mg.

It is particularly preferred to use thiazide diuretic and loop diuretic in amounts and proportions which, for the duration of action of the combination, i.e. during the period of time within which NaCl excretion induced by the active ingredients is above control excretion without ingredient combination (“baseline”), results in a K⁺/Na⁺ excretion ratio of ≦0.3. Such a K⁺/Na⁺ excretion ratio is achieved, for example, by a combination of about 20 to 30 mg of hydrochlorothiazide and about 7.5 to 12.5 mg of torasemide, for example by an ingredient combination of 25 mg of hydrochlorothiazide and 10 mg of torasemide, or of corresponding amounts of other thiazide and loop diuretics.

The combination preparation according to the invention, as mentioned above, may contain the active ingredients either jointly or separately from each other, i.e. in the form of a sole dosage unit or of separate dosage units. Preferably, the combination preparation of the present invention is present in the form of a sole dosage unit, more preferably in the form of a solid pharmaceutical composition which contains both active ingredients. It is useful for oral administration, for example in the form of powder, granules, tablets, coated tablets and capsules.

The combination preparation of the present invention, optionally, may contain the active ingredients, jointly or separately from each other, in admixture with at least one pharmaceutically acceptable excipient or additive. Suitable excipients and additives are, for example, fillers such as sucrose, lactose, cellulose such as microcrystalline cellulose, mannitol, maltitol, dextrane, starches like corn starch, agar, alginates, chitins, chitosanes, pectins, gum tragacanth, gum arabic, gelatine, caseine, albumine, synthetic and semi synthetic polymers or glycerides, lubricants such as magnesium stearate, preservatives such as paraben or sorbic acid, antioxidants such as ascorbic acid, α-tocopherol or cysteine, flow regulators and desiccants, for example highly dispersed silicon dioxide, disintegrants, for example sodium carboxymethyl starch, binders, thickening agents, sweeteners or flavouring agents.

The combination preparations of the present invention can be prepared in a manner known by a person skilled in the art. For example, compositions containing the active ingredients jointly or separately may be prepared by thoroughly mixing and combining the active ingredients and, optionally, the pharmaceutically acceptable excipients and additives in powder form.

The fixed combination of thiazide diuretic and loop diuretic according to the invention is particularly useful for the treatment of cardiovascular diseases such as hypertension and heart failure. The present invention thus also relates to the use of a thiazide diuretic and a loop diuretic as active ingredients for the manufacture of a combination preparation for the treatment of hypertension or heart failure.

The combination of thiazide diuretic and loop diuretic according to the invention is particularly useful for the treatment of primary (essential) hypertension.

In addition, secondary hypertension, for example renal hypertension, may be well treated using the combination of the present invention, particularly in case of only a slight or moderate renal insufficiency with a GFR≧30 ml/min (plasma creatinine≦2 mg/dl), which consistently occurs in this group of patients.

The combination of thiazide diuretic and loop diuretic according to the invention, in addition, is very useful for treatment of chronic and subchronic forms of heart failure with unreduced effective arterial blood volume (EABV) and a cardiac index (cardiac output per body surface [l min⁻¹m⁻²]) of ≧2 l min⁻¹m⁻² (according to 32 and 33), as well as for stabilizing the heart after recompensation of acute heart failure.

The combination preparation according to the invention preferably is administered to the subject in need of such treatment, in particular humans, once or twice a day, more preferably once a day. The precise dosage for a patient depends, for example, on the patient's age, gender, body weight and diet and on the patient's general condition and the condition to be treated. The precise dosage can be easily determined by the skilled physician by considering these and other factors.

Optionally, the combination preparation of thiazide diuretic and loop diuretic according to the invention can be co-administered with other pharmaceuticals useful for treating hypertension or heart failure. Examples for those pharmaceuticals are ACE inhibitors such as captopril, benazepril and enalapril, and AT₁ receptor antagonists such as candesartan, irbesatan and valsatan.

The combination preparations of the present invention have the advantage that there are no high peaks in efficacy (peak diuresis) and, unlike single dose administrations of loop diuretics, there is no pronounced rebound, i.e., there is no post diuretic period where NaCl excretion is below baseline excretion. The duration of action of the loop diuretic is thus prolonged by the thiazide resulting in a favourable daily profile allowing single dose administration, which is particularly desired for hypertensive patients. As a further advantage, the combination preparation of thiazide diuretic and loop diuretic, normalized with respect to the NaCl excretion, surprisingly is significantly improved with regard to the excretion of potassium and magnesium when compared to the conventional combination of thiazide and antikaliuretic. At the same time, the desired hypocalciuric effect of the thiazide is maintained despite the presence of the loop diuretic. This effectively allows preventing undesired hypokalemia, hypomagnesemia and/or hypocalcemia and secondary effects associated therewith such as amyasthenia, obstipation, osteoclasis and cardiac arrhythmias. In addition, the combination allows co-administering of an ACE inhibitor or of an AT₁ antagonist.

Thus, the combination preparation of active ingredients according to the invention provides drugs giving rise to an effective natriuresis with only minor potassium and magnesium losses and thus having an improved profile of benefit/secondary effects in addition to a good efficacy. The combination preparation of active ingredients, thus, is extremely useful for long-term and permanent treatment.

The present invention is described in the following in more detail with reference to examples and figures.

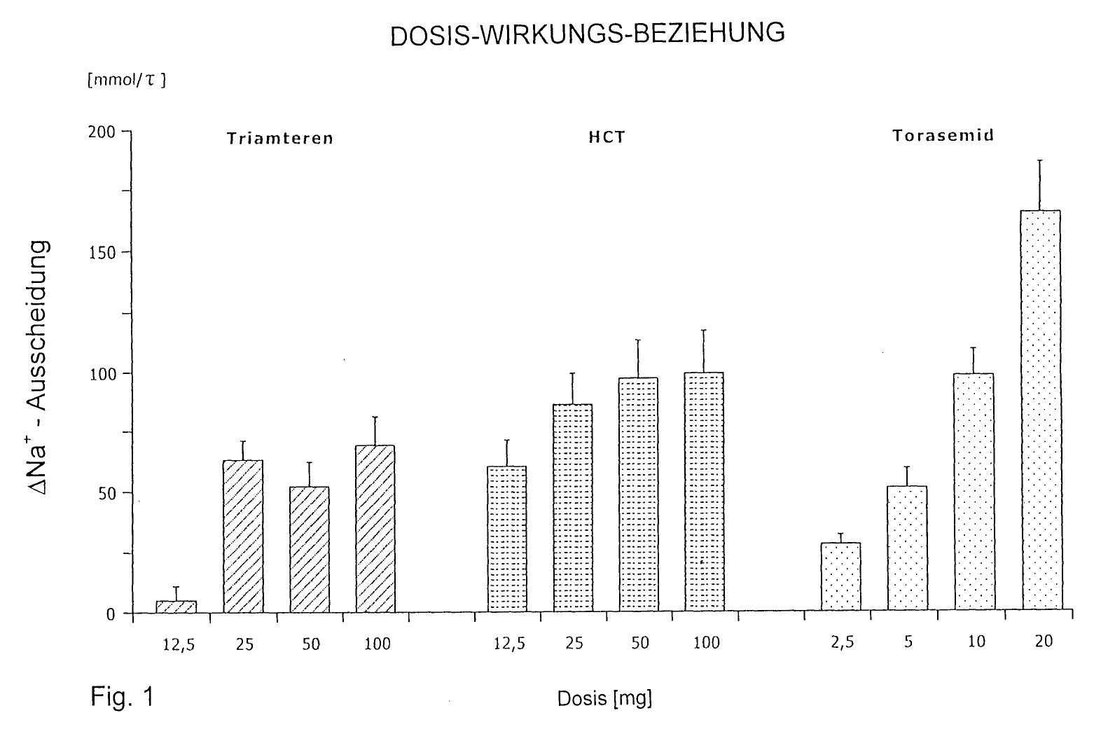

FIG. 1 shows the net Na⁺ excretion (ΔNa⁺) (=total excretion minus baseline excretion as determined within the same period of time the day before) for the duration of action (τ) (=time where drug-induced excretion exceeds baseline excretion) in dependency on the administered dose of triamterene, hydrochlorothiazide (HCT) and torasemide;

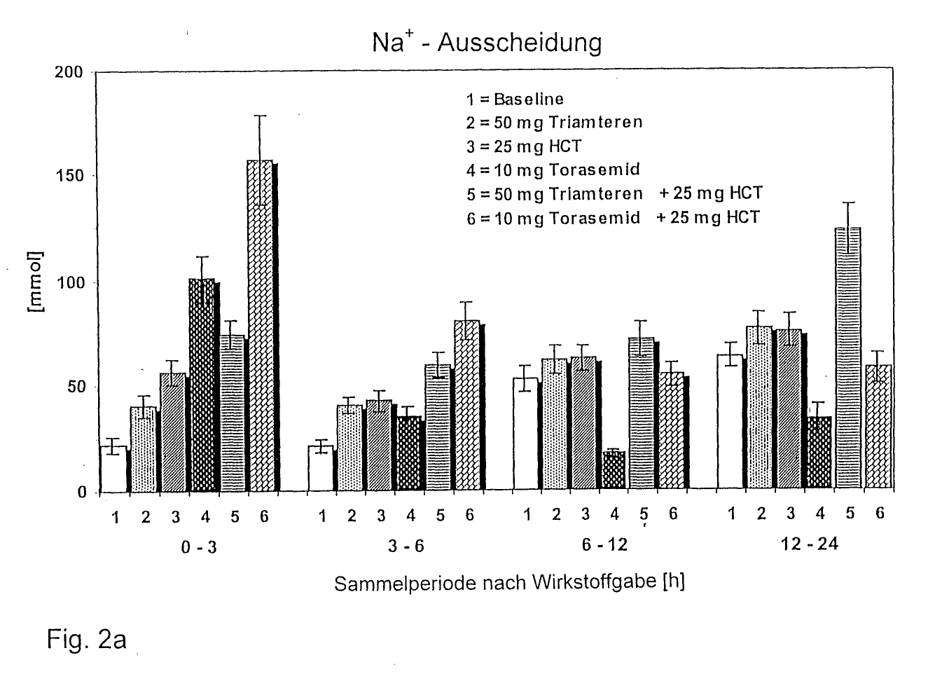

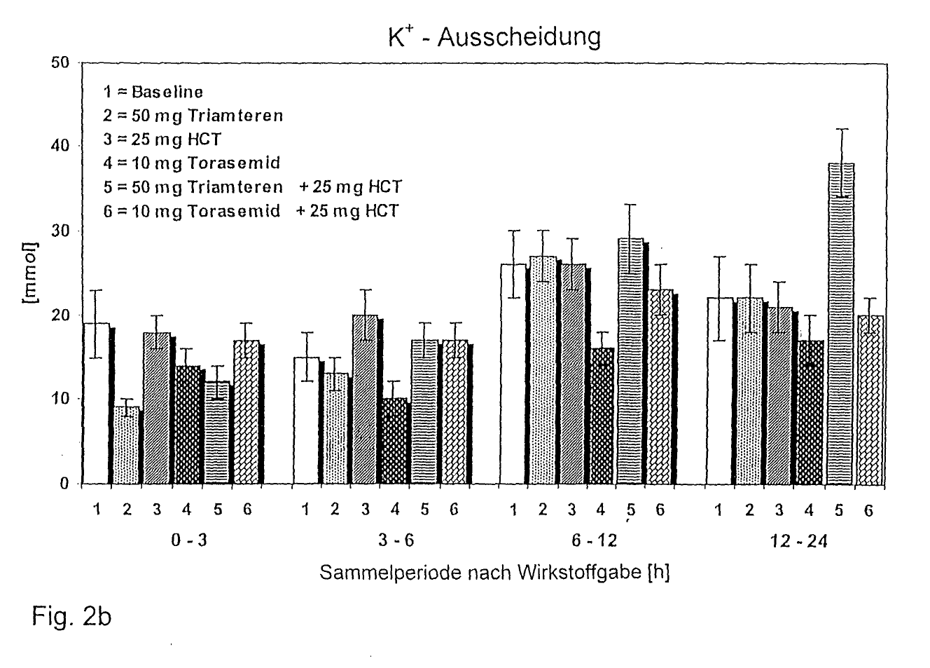

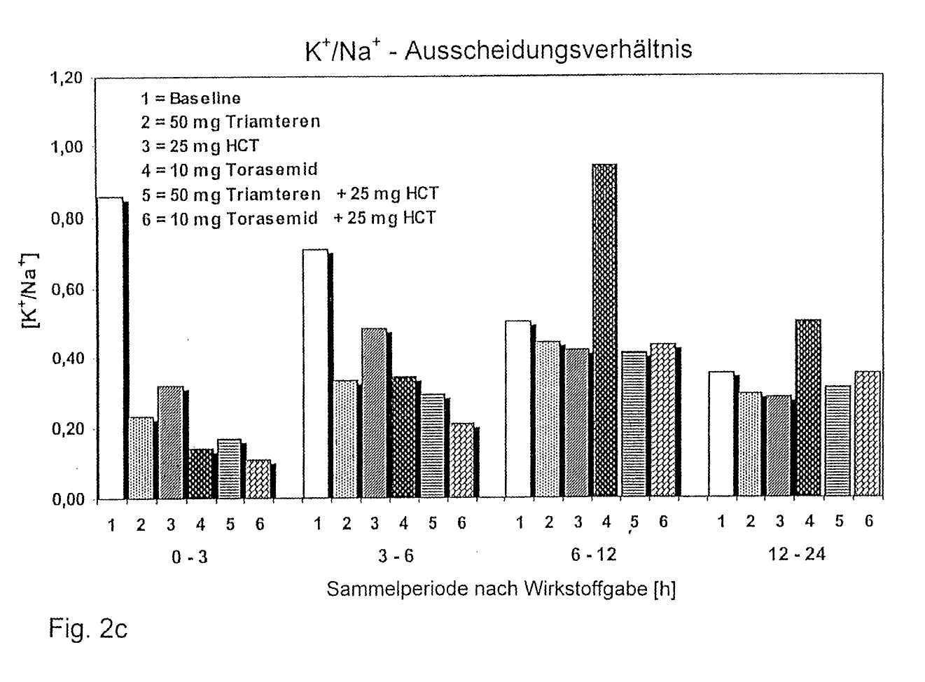

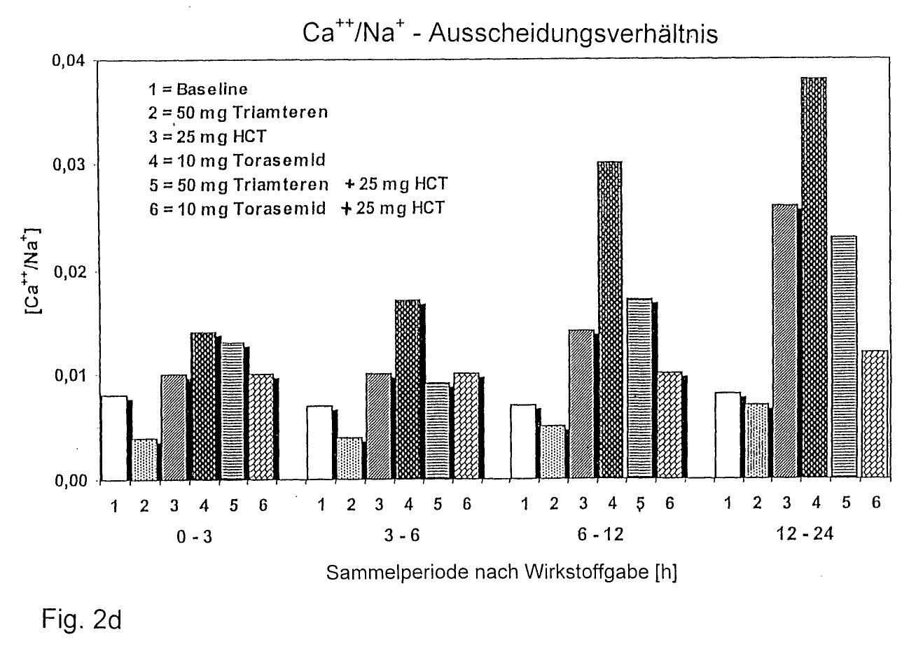

FIG. 2a-d show the time profile of Na⁺ excretion (2a) and K⁺ excretion (2b) cumulated for distinct periods after drug administration, as well as the excretion ratio of K⁺/Na⁺ (2c) and Ca²⁺/Na⁺ (2d) in urine for the respective collection period;

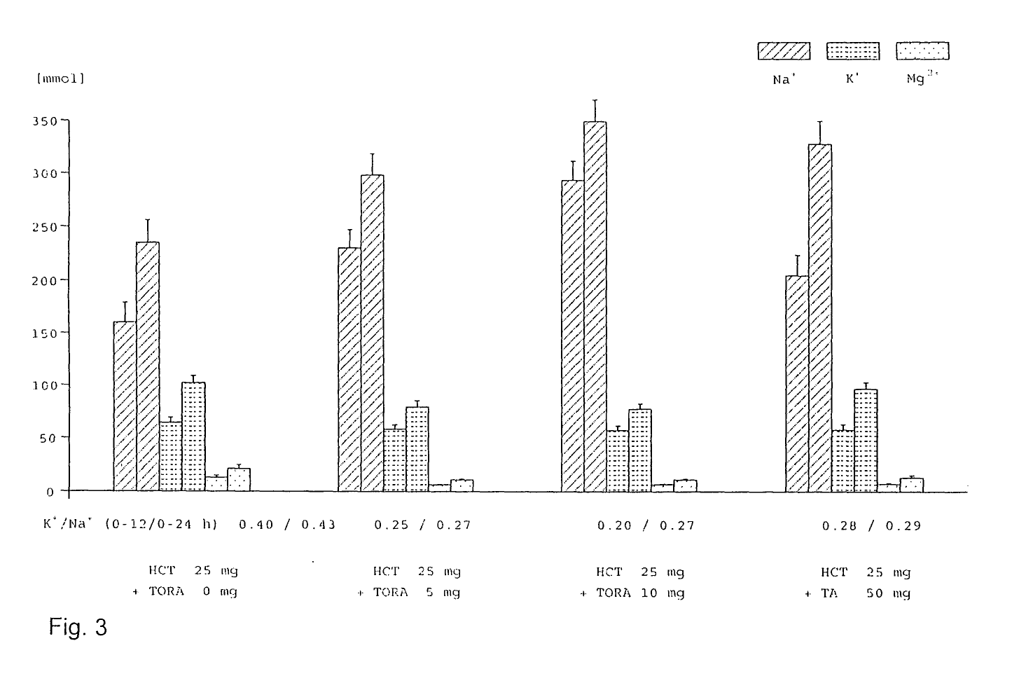

FIG. 3 shows the total cumulative excretion of Na⁺, K⁺ and Mg²⁺ for the 0-12 hrs period (left bar of the pair of bars) and the 0-24 hrs period (right bar) following administration of hydrochlorothiazide (HCT) and torasemide (TORA) compared to HCT-monotherapy. Data obtained using a conventional combination of HCT and triamterene (TA) are given for comparison. The Na⁺/K⁺ excretion ratios, as determined for 0-12 hrs (0-24 hrs) following administration of the respective regime, are indicated below the bars.

### EXAMPLES

### 1. Methods

The ethically approved trial was performed on 12 healthy volunteers (28-48years in age, 6 males, 6 females), who had given their written informed consent to the study. Prior to administration of a drug, the participants underwent a clinical check-up. The participants were kept on a standard diet, receiving a defined fluid (1.5 l/day) and salt intake (6 g NaCl; 100 mmol Na⁺/day) three days before and throughout the study.

In accordance with earlier studies (35), urine was collected before the treatment period, i.e., from 24 hrs to 12 hrs and from 12 hrs to 0 hrs prior to oral drug administration (8 a.m.). From that point of time, urine was collected following drug administration in 2 hour intervals until the 6^{th} hour and then from the 6^{th} to 9^{th} hour, from the 9^{th} to 12^{th} hour and from the 12^{th} to 4^{th} hour. An aliquot of the defined volume of the respective collection period was frozen until analyzed for Na⁺, K⁺, Cl⁻, Ca²⁺ Mg²⁺ and creatinine using a “MODULAR” Analyzer (Roche Comp., Switzerland).

All data are expressed as mean values (±SEM). The diuretic-induced “net excretion” is defined as the difference between total excretion and control excretion (“baseline”) obtained for the respective period prior to treatment. The effect of diuretic monotherapy is compared with the combined treatment using ANOVA.

### 2. Results

### A. Dose-Response Studies

Dose-response studies of hydrochlorothiazide (HCT) and triamterene (TA) confirm well known findings that these classes of diuretics reach their maximum natriuretic effect already with commonly used doses of 25 mg of HCT and 50 mg of TA, respectively (FIG. 1). Yet, cumulative Na⁺ excretion induced by 25 mg of HCT during 12 hrs (the time until baseline excretion is reached again) was not significantly different from that obtained with 50 mg. The narrow range of almost linear dose-responsiveness characterizes these diuretics as “low ceiling” diuretics.

The “high ceiling” loop diuretic torasemide (TORA), on the other hand, has a broader range of linear dose-responsiveness in relation to its short duration of action (6 hrs). But this class of diuretics is known to be significantly less effective than thiazide when the natriuretic effect of one daily dose is based on 24 hrs (33, 34).

### B. Diuretic Effects on the Excretion of Na⁺, K⁺, Mg²⁺ and Ca²⁺.

### 1. Monotherapy

The natriuretic effect of usual doses of diuretics then was correlated with the excretion of K⁺, Mg²⁺ and Ca²⁺, defined for the different periods after drug administration. FIG. 2a shows that 10 mg of TORA resulted by far in the highest increase of Na excretion during the first 3 hrs, followed by 25 mg of HCT and then by 50 mg of TA. During the 3-6 hrs period, this difference disappeared und during the 6-12 hrs period and the 12-24 hrs period the loop diuretics showed the well known “rebound”, i.e., the Na⁺ excretion dropped below levels prior to drug administration. HCT induced a higher Na⁺ excretion than TA only during the first 3 hrs.

The HCT-induced total K⁺ excretion was higher than that following administration of the loop diuretic. This was true in particular for the duration of action of the thiazide (0-12 hrs) and for its post-diuretic period (12-24 hrs). TA induced less K⁺ loss than HCT or TORA only for the 0-3 hrs period (FIG. 2b).

When K⁺ excretion—as defined previously (35, 36) —was correlated with Na⁺ excretion it became evident that the K⁺/Na⁺ excretion ratio of HCT within the first three hours following drug administration was nearly triple of that of the loop diuretic (FIG. 2c). The K⁺/Na⁺ excretion ratio of TORA was highest only after the duration of action of the loop diuretic (6-24 hrs), because then Na⁺ excretion was below baseline. Surprisingly, the potassium sparing diuretic TA was not superior to the loop diuretic neither with respect to total K⁺ excretion (0-24 hrs) (FIG. 2b) nor to Na⁺ excretion in relation to K⁺ excretion (0-6 hrs) (FIG. 2c).

Mg²⁺ excretion did not change significantly in relation to that of K⁺ following administration of thiazide and loop diuretic (FIG. 3). In other words, the loop diuretic is less magnesiuretic than the thiazide. On the other hand and in agreement with previous observations (35, 36), Ca²⁺ excretion in relation to Na⁺ excretion was higher for the loop diuretic than for the thiazide (FIG. 2d).

### 2. Combinations of Diuretics

Co-administration of HCT and loop diuretic or potassium sparing diuretic significantly increased the natriuretic effect for the 0-6 hrs period (FIG. 2a). The HCT/TORA combination resulted in a supra-additive natriuretic effect, i.e., net Na⁺ excretion (total excretion minus baseline) for the co-administered diuretics combination was significantly higher than the sum of each class of diuretics when administered alone. If at all, HCT in combination with the potassium sparing diuretic only induced an additive natriuretic effect.

Most surprising was excretion of K⁺, Mg²⁺ and Ca²⁺ in relation to the natriuretic effect of the diuretics combination. As shown in FIG. 3, total excretion of K⁺ and Mg²⁺, for the duration of action (0-12 hrs) as well as for the period of 0-24 hrs, was less for co-administration with TORA than for HCT given alone. This difference is best quantified by correlating K⁺ excretion with Na⁺ excretion, which was reduced by 5 mg TORA from 0.40 (0.43) to 0.25 (0.27) (p<0.01). This “K⁺ sparing effect” of TORA was even more pronounced for co-administration of 10 mg TORA with HCT (FIG. 3).

Co-administration of TORA also reduced HCT-induced excretion of Mg²+ (p<0.01). Moreover, from the time course of electrolyte excretion shown in FIG. 2 it can be seen that the hypocalciuric effect of HCT is maintained when combined with the loop diuretic (FIG. 2d).

By comparing the HCT/TORA combination with a conventional HCT/TA combination (FIG. 3) it becomes evident that the loop diuretic is superior to the potassium sparing diuretic compared to administration of thiazides alone, and this holds true with regard to the desired natriuretic effect as well as to the undesired K⁺ and Mg²⁺ losses.

### REFERENCES

1. Black W D, Shiner P T, Roman J. (1978) Severe electrolyte disturbances associated with metolazone and furosemide. South Med J 71: 381-385
2. Leiter L (1970) Combinations of diuretics in the treatment of edema. Am Heart J 80: 422
3. Gunstone R F, Wing A J, Shani H G P, Njemo D, Sabuka E M W (1971) Clinical experience with metolazone in fifty-two African patients: synergy with furosemide. Postgrad med J 47: 789-793
4. Oleson K H (1971) The natriuretic effect of addition of quinethazone and furosemide in congestive heart failure. Acta Med Scand 190:229
5. Olesen K H, Sigurd B (1971) The supra-additive natriuretic effect by addition of quinethazone or bendroflumethiazide during long-term treatment with furosemide and spironolactone: permutation trial tests in patients with congestive heart failure. Acta Med Scand 199: 233-240
6. Asscher A W (1974) Treatment of frusemide resistant oedema with metolazone. Clin Trials J 4: 134-138
7. Sigurd B, Olesen K H, Wennevold A (1975) The supra-additive natruretic effect of addition of bendroflumethiazide and bumetanide in congestive heart failure. Am Heart J 86; 163-170
8. Ram C V S, Reichgott M J (1977) Treatment of loop diuretic resistant edema by the addition of metolazone. Curr Ther Res 22: 686-691
9. Epstein M, Lepp B A, Hoffman D S, Levinson R (1977) Potentiatioin of furosemide by metolazone in refractory edema. Current Therapeutic Research 21:656-667
10. Furrer, J Hess O M, Kuhlmann U, Satz N, Siegenthaler W (1980) Furosemid und Metolazon: eine hochwirksame Diuretikakombination Schweiz Med Wschr 110: 1825-1829
11. Garin E H, Richard G A (1981) Edema resistant to furosemide and metolazone. Int J Pediatr Nephrol 2: 181-184
12. Ghose R R, Gupta S K (1981) Synergistic action of metolazone with loop diuretics. Br Med J 282: 1825-1829
13. Wollam G Tarazi R., Bravo E. and Dustan H. (1982) Diuretic potency of combined hydrochlorothiazide and furosemide therapy in patients with azotemia. Am J Med 72, 929-938
14. Oster J R, Epstein M, Smoller S (1983) Combined therapy with thiazide-type and loop diuretic agents for resistant sodium retention. Ann Intern Med 99: 405-406
15. Arnold W C (1984) Efficacy of metolazone and furosemide in children with furosemid resistant edema. Pediatrics 74: 872-875
16. Marone C., Muggli F., Lahn W., and Frey F. J. (1985) Pharmacokinetic and pharmacodynamic interaction between furosemide and metolazone in man. Eur J Clin Invest. 15: 253-257
17. Garin E H (1987) A comparison of combinations of diuretics in nephrotic edema. AJDD 141: 769-771
18. Channer K S, Richardson M, Crook R, Jones J V (1990) Thiazides with loop diurectis for severe congestive heart failure. Lancet 335: 922-923
19. Oimomi M, Takase S, and Saeki S (1990) Combination diuretic therapy for severe refractory nephrotic syndrome. Lancet 336: 1004-1005
20. Knauf H, Mutschler E (1993) Low dose segmental blockade of the nephron rather than high dose diuretic monotherapy. In “Diuretics IV. Chemistry Pharmacology and Clinical Applications” (J. Puschett and A. Greenberg, Eds.), pp 449-456. Elsevier Amsterdam.
21. Channer K S, McLean K., Lawson-Matthew P, and Richardson M (1994). Combination diuretic treatment in severe heart failure: A randomised controlled trial. Br Heart J 71: 146-150
22. Fliser D, Schroter M, Neubeck M, and Ritz E. (1994) Coadministration of thiazides increases the efficacy of loop diuretics even in patients with advanced renal failure. Kidney Int. 46: 482-488
23. Knauf H, Mutschler E (1995) Diuretic effectiveness of hydrochlorothiazide and furosemide alone and in combination in chronic renal failure. J Cardiovasc Pharmacol 26: 394-400
24. Ellison D H (1991) The physiologic basis of diuretic synergism: Its role in treating diuretic resistance. Ann Int Med. 114: 886-894
25. Brater D C (1994) Diuretic Resistance: Mechanisms and Therapeutic Strategies. Cardiology 84 (suppl 2): 57-67
26. Kaissling B, Bachmann S, and Kriz W (1985) Structural adaptation of the distal convoluted tubule to prolonged furosemide treatment. Am J Physiol 248, F374-F381
27. H. Knauf et al. (1997) Therapieempfehlungen für Patienten mit “diuretikaresistenten” hydropischen Zuständen. Notabene Medici 7, 350-351
28. Anderson J, Godfrey B E, Hill D M, Munro-Faure A D, Sheldon J (1971) A comparison of the effects of hydrochlorothiazide and of furosemide in the treatment of hypertensive patients. Quarterly Journ of Med XL: 541-560
29. Bariso C R, Hanenson I B, Gaffney T E (1970) A comparison of the antihypertensive effects of furosemide and chlorothiazide. Curr Ther Res 12: 333-340
30. McMahon F G (1990) Management of essential hypertension. The once-a-day era. Futura, New York, S. 287-378
31. Knauf H und Mutschler E (1996) Das Wirkprofil von Diuretika. Nieren- und Hochdruckkrankheiten 25(4): 162-170
32. Roskamm H, Reindel H (1982) Herzkrankheiten. Springer Verlag Berlin, S. 361
33. Buchwalsky R (1985) Einschwemmkatheter. In: Beiträge zur Kardiologie Band 29, S. 143, perimed Fachbuch-verlagsgesellschaft Erlangen
34. Leary W P und Reyes A J (1993) Low-dose segmental blockade of the nephron rather than high-diuretic monotherapy. In: Diuretics IV. Chemistry Pharmacology, and Clinical Applications. Puschett J, Greenberg A (Hrsg.) Elsevier, Amsterdam, S. 449-456
35. Knauf H und Mutschler E (1990) Saluretic effects of the loop diuretic torasemid in chronic renal failure. Interdependence of electrolyte excretion. Eur J Clin Pharmacol 39: 337-343
36. Velazquez H, Knauf H und Mutschler E (1995) Thiazide diuretics. In: Diuretics. Greger R F, Knauf H, Mutschler E (Hrsg.), Springer, Berlin, New York, S. 275-334

1. A combination preparation, comprising, jointly or separately from each other, a thiazide diuretic and a loop diuretic, wherein the thiazide diuretic is selected from the group consisting of:
hydrochlorothiazide in an amount of from 5 to 50 mg;
hydroflumethiazide in an amount of from 5 to 50 mg;
benzthiazide in an amount of from 5 to 50 mg;
chlorthalidone in an amount of from 5 to 50 mg;
quinethazone in an amount of from 5 to 50 mg;
bendroflumethiazide in an amount of from 0.5 to 5 mg;
methychlothiazide in an amount of from 0.5 to 5 mg;
indapamide in an amount of from 0.25 to 1.25 mg;
polythiazide in an amount of from 0.2 to 2 mg;
trichlormethazide in an amount of from 0.2 to 2 mg;
and the loop diuretic is selected from the group consisting of:
torasemide in an amount of from 2.5 to 15 mg;
piretanide in an amount of from 2.5 to 15 mg;
furosemide in an amount of from 7.5 to 37.5 mg; and
bumetanide in an amount of from 0.05 to 0.3 mg.
and from pharmaceutically acceptable derivatives of said thiazide diuretics and loop diuretics.

2. The combination preparation according to claim 1, wherein the thiazide diuretic is hydrochlorothiazide and the loop diuretic is torasemide.

3. The combination preparation according to claim 1 in the form of a solid pharmaceutical composition comprising the thiazide diuretic and the loop diuretic.

4. The combination preparation according to claim 1, wherein the thiazide diuretic and the loop diuretic are present separated from each other in the combination preparation.

5. The combination preparation according to claim 1 further comprising at least one pharmaceutically acceptable excipient or additive.

6. The combination preparation according to claim 5, wherein the pharmaceutically acceptable excipient or additive is selected from the group consisting of fillers, lubricants, preservatives, antioxidants, disintegrants, binders, thickening agents, sweeteners and flavouring agents.

7. The combination preparation according to claim 1 wherein the combination preparation is in the form of powder, granules, tablets, coated tablets or capsules.

8. The combination preparation according to claim 1, wherein the thiazide diuretic is selected from the group consisting of:
hydrochlorothiazide in an amount of from 10 to 30 mg;
hydroflumethiazide in an amount of from 10 to 30 mg;
benzthiazide in an amount of from 10 to 30 mg;
chlorthalidone in an amount of from 10 to 30 mg;
quinethazone in an amount of from 10 to 30 mg;
bendroflumethiazide in an amount of from 1 to 3.0 mg;
methychlothiazide in an amount of from 1 to 3.0 mg;
indapamide in an amount of from 0.5 to 1.5 mg;
polythiazide in an amount of from 0.4 to 1.2 mg;
trichlormethazide in an amount of from 0.4 to 1.2 mg;
and the loop diuretic is selected from the group consisting of:
torasemide in an amount of from 2.5 to 12.5 mg;
piretanide in an amount of from 2.5 to 12.5 mg;
furosemide in an amount of from 7.5 to 37.5 mg; and
bumetanide in an amount of from 0.05 to 0.3 mg.
and from pharmaceutically acceptable derivatives of said thiazide diuretics and loop diuretics.

9. The combination preparation according to claim 8, wherein the thiazide diuretic is hydrochlorothiazide and the loop diuretic is torasemide.

10. The combination preparation according to claim 8 in the form of a solid pharmaceutical composition comprising the thiazide diuretic and the loop diuretic.

11. The combination preparation according to claim 8, wherein the thiazide diuretic and the loop diuretic are present separated from each other in the combination preparation.

12. The combination preparation according to claim 8 further comprising at least one pharmaceutically acceptable excipient or additive.

13. The combination preparation according to claim 12, wherein the pharmaceutically acceptable excipient or additive is selected from the group consisting of fillers, lubricants, preservatives, antioxidants, disintegrants, binders, thickening agents, sweeteners and flavouring agents.

14. The combination preparation according to claim 8 wherein the combination preparation is in the form of powder, granules, tablets, coated tablets or capsules.

15. A method of using a combination preparation of a thiazide diuretic and a loop diuretic for the treatment of hypertension or heart failure, wherein the thiazide diuretic is selected from the group consisting of:
hydrochlorothiazide in an amount of from 5 to 50 mg;
hydroflumethiazide in an amount of from 5 to 50 mg;
benzthiazide in an amount of from 5 to 50 mg;
chlorthalidone in an amount of from 5 to 50 mg;
quinethazone in an amount of from 5 to 50 mg;
bendroflumethiazide in an amount of from 0.5 to 5 mg;
metolazone in an amount of from 0.5 to 5 mg;
methychlothiazide in an amount of from 0.5 to 5 mg;
indapamide in an amount of from 0.25 to 1.25 mg;
polythiazide in an amount of from 0.2 to 2 mg;
trichlormethazide in an amount of from 0.2 to 2 mg;
and the loop diuretic is selected from the group consisting of:
torasemide in an amount of from 2.5 to 15 mg;
piretanide in an amount of from 2.5 to 15 mg;
furosemide in an amount of from 7.5 to 45 mg; and
bumetanide in an amount of from 0.05 to 0.3 mg.
and from pharmaceutically acceptable derivatives of said thiazide diuretics and loop diuretics.

16. The method according to claim 15 wherein the combination preparation is for treatment of primary hypertension.

17. The method according to claim 15 wherein the combination preparation is for treatment of renal hypertension with plasma creatinine≦2 mg/dl.

18. The method according to claim 15 wherein the combination preparation is for the treatment of heart failure with unreduced effective arterial blood volume (EABV).

19. The method according to claim 15 wherein the method further comprises treatment with an ACE inhibitor or an AT1 receptor antagonist.

20. The method according to claim 15 wherein the method is for long-term therapy or permanent therapy.
